(19) United States
(12) Patent Application Publication
(10) Pub. No.: US 2011/0236391 A1
(43) Pub. Date: Sep. 29, 2011

(54) METHOD FOR OBTAINING AN EXCIPIENT-FREE ANTIBODY SOLUTION

(76) Inventors: Hanns-Christian Mahler, Basel (CH); Robert Mueller, Basel (CH)

(21) Appl. No.: 13/132,809

(22) PCT Filed: Dec. 3, 2009
(86) PCT No.: PCT/EP2009/066329

§ 371 Date: Jun. 3, 2011

(30) Foreign Application Priority Data
Dec. 9, 2008 (EP) 08171023.8

(51) Int. Cl. A61K 39/395

(52) U.S. Cl. 424/141.1; 424/130.1

(57) ABSTRACT

The present invention relates to a method of ultra- and dialfiltrating an antibody solution containing at least one solute in addition to the antibody, which comprises diafiltrating the antibody solution with a solvent and bringing said mixture into contact with a semi-permeable membrane so as to allow the at least one solute present in the antibody solution and having a molecular weight lower than the molecular weight cut-off of the membrane to pass through the membrane, whilst retaining the antibody so that a modified antibody solution is obtained that only contains the antibody and the solvent.

The present invention relates to a method for obtaining an excipient-free antibody solution by ultrafiltration-diafiltration.

Antibody molecules, as part of the group of protein pharmaceuticals, are very susceptible to physical and chemical degradation, such as denaturation and aggregation, deamidation, oxidation and hydrolysis. Protein stability is influenced by the characteristics of the protein itself, e.g. the amino acid sequence, and by external influences, such as temperature, solvent pH, excipients, interfaces, or shear rates. So, it is important to define the optimal formulation conditions to protect the protein against degradation reactions during manufacturing, storage and administration. (Manning, M. C., K. Patel, et al. (1989). “Stability of protein pharmaceuticals.” Pharm Res 6(11): 903-18, Zheng, J. Y. and L. J. Janis (2005). “Influence of pH, buffer species, and storage temperature on physicochemical stability of a humanized monoclonal antibody LA298.” Int J. Pharm.)

Administration of antibodies via subcutaneous (s.c.) or intramuscular (i.m.) route requires high protein concentrations in the final formulation due to the often required high doses and the limited administration volumes of the s.c. or i.m. route. (Shire, S. J., Z. Shahrokh, et al. (2004). “Challenges in the development of high protein concentration formulations.” J Pharm Sci 93(6): 1390-402, Roskos, L. K., C. G. Davis, et al. (2004). “The clinical pharmacology of therapeutic monoclonal antibodies.” Drug Development Research 61(3): 108-120.) The large-scale manufacturing of high protein concentration can be achieved by ultrafiltration processes, drying process, such as lyophilisation or spray-drying, and precipitation processes. (Shire, S. J., Z. Shahrokh, et al. (2004). “Challenges in the development of high protein concentration formulations.” J Pharm Sci 93(6): 1390-402.)

Andya et al. (U.S. Pat. No. 6,267,958, U.S. Pat. No. 6,85,940) describe a stable lyophilized formulation of an antibody, which is reconstituted with a suitable diluent volume to achieve the required concentration. The formulation comprises a lyoprotectant, a buffer and a surfactant.

Membrane filtration is a technique widely used in the life sciences, most commonly for the separation, purification or concentration of proteins. Depending on membrane type it can be classified as microfiltration (membrane pore size between 0.1 and 10 μm) or ultrafiltration (membrane pore size between 0.001 and 0.1 μm). Ultrafiltration membranes are used for concentrating dissolved molecules (protein, peptides, nucleic acids, carbohydrates, and other biomolecules), desalting or exchanging buffers, and gross fractionation. An ultrafiltration membrane retains molecules that are larger than the pores of the membrane, while smaller molecules such as salts, solvents and water, which are 100% permeable, freely pass through the membrane. There are two main membrane filtration methods: in Single Pass/Dead End/Direct Flow Filtration pFF), the fluid to be filtered is directed perpendicular to the membrane. In Cross Flow/Tangential Flow Filtration (TFF), the fluid flows tangential to the surface of the membrane; TFF solves the problem of membrane clogging by re-circulating the retentate.

In macromolecular concentration, the membrane enriches the content of a desired biological species. Pressure, created by external means, forces liquid through the semi-permeable membrane. Solutes larger than the nominal molecular weight cut-off (MWCO) of the membrane are retained. The required pressure can be generated by use of compressed gas, pumping, centrifugation or capillary action.

Removal of small molecules from a solution by alternating ultrafiltration and re-dilution or by continuous ultrafiltration and dilution to maintain constant volume leads to a diafiltration process. Ultrafiltration is ideal for removal or exchange of salt, sugars, non-aqueous solvents or rapid change of ionic and pH environment.

In general, an ultrafiltration installation encompasses a feed solution containing, a macromolecule (e.g. an antibody), solutes, such as buffer components, salts, amino acids or sugars and solvent (e.g. water) is forced by external forces (e.g. by pumping) through an ultrafiltration cassette. The feed stream is separated into a filtrate and retentate stream. The filtrate consists of the solvent and all solutes, which are able to pass the semi-permeable membrane, and leaves the system circulation. The macromolecule is retained in the retentate stream and is returned to the feed tank. During a concentration process the solvent is constantly removed and the macromolecule concentration is increased, whereas the concentration of solutes, which are able to pass the membrane, remains constant. During a diafiltration process, the discharging filtrate volume is compensated by adding diafiltration buffer to the feed tank. The diafiltration buffer consists of a different composition of solutes than the original feed solution. The concentration of the macromolecule remains constant, whereas the solute composition changes constantly from the initial feed composition to the composition of the diafiltration buffer. Both processes, concentration and diafiltration, can be combined in variable sequences.

U.S. Pat. No. 6,566,329 describes the manufacturing of freeze-dried preparations of human growth hormone, where desalting of hGH was performed as an intermediate process steps using a desalting column to obtain hGH in pure water without salts and other excipients. The scope of this work was to develop a freeze-dried preparation and it is limited to hGH at a lower solubility of maximum 70 mg/mL concentration and a desalting column was used.

WO 99/55362 teaches spray-dried formulations of IGF-1. Pure rhIGH-1 was employed as one intermediate for its preparation. The buffer exchange, however, was performed using dialysis cassettes and pure IGF-1 in water was obtained, which showed strong turbidity and precipitation, i.e. strong signs of instability, and the solubility of IGF-1 in water was markedly reduced compared to excipient-containing formulations with a maximum of 24 mg/mL.

Gokarn et al., J. Pharm. Sci. 2007 Nov. 19; 97(8): 3051-3066 showed the self-buffering capacity of high-concentration antibody formulations, so demonstrating the possibility to exclude buffer components from the protein formulation. However, the addition of sorbitol to the buffer-free preparations was necessary to ensure stability and isotonicity of the described antibody formulations. In WO2006/138181, Gokarn et al also described the self-buffering capacity of high-concentration antibody formulations, which included a brief description of a process for preparation a buffer-free composition removing residual buffer using size-exclusion chromatography, dialysis and/or tangentional flow filtration (ultrafiltration-diafiltration), however, solely in the presence of a counter ion.

The objective of the invention was to develop a method for the preparation of an excipient-free antibody solution that does not have the disadvantages of the prior art or at least partially avoid these disadvantages.

This objective is achieved by the method in accordance with the independent claims. An antibody solution containing various solutes, such as buffer salts, salts, amino acids, sugars or sugar alcohols is buffer-exchanged against pure water by diafiltration, resulting in a solution consisting only of the antibody and the solvent. Optionally, concentration steps can be added before and after the diafiltration step. Surprisingly, the so-obtained excipient-free protein formulation of an antibody sustains overall protein stability during diafiltration and concentration.

The first aspect of the invention concerns a method of ultra- and diafiltrating an antibody solution containing at least one solute in addition to the antibody, which comprises diafiltering the antibody solution with a solvent and bringing said mixture into contact with a semi-permeable membrane so as to allow the at least one solute present in the antibody solution and having a molecular weight lower than the molecular weight cut-off (MWCO) of the membrane to pass through the membrane, whilst retaining the antibody so that a modified antibody solution is obtained that only contains the antibody and the solvent. Preferably, said solvent is water and the at least one solute is selected from the group consisting of buffer salts, salts, amino acids, sugars and sugar alcohols.

Examples of antibodies that are useful in the present invention are immunoglobulin molecules, e.g. IgG molecules. IgGs are characterized in comprising two heavy and two light chains and these molecules comprise two antigen binding sites. Said antigen binding sites comprise “variable regions” consisting of parts of the heavy chains (VH) and parts of the light chains (VL). The antigen-binding sites are formed by the juxtaposition of the VH and VL domains. For general information on antibody molecules or immunoglobulin molecules see also common textbooks, like Abbas “Cellular and Molecular Immunology”, W.B. Sounders Company (2003).

The method of ultra- and diafiltrating an antibody solution containing at least one solute in addition to the antibody as described hereinbefore preferably leads to an excipient-free antibody solution with an antibody concentration of from 30 to 280 mg/mL, and more preferably of from 80 to 200 mg/mL.

The method described hereinbefore can be used to manufacture final products either in liquid or dried form. Consequently, the inventive method further comprises the step of processing said antibody solution that only contains the antibody and the solvent to a lyophilizate, stable liquid formulation and/or reconstituted formulation.

The antibody is preferably a monoclonal antibody and especially preferred are monoclonal antibodies selected from the group of IgG1, IgG2 or IgG4.

The second aspect of the invention concerns a purified antibody solution obtainable by the inventive method. Preferably, said antibody is a monoclonal antibody, even more preferred is when said antibody is a monoclonal antibody selected from the group if IgG1, IgG2 or IgG4

The third aspect of the invention concerns a lyophilized antibody preparation obtained by lyophilizing the inventive purified antibody solution as mentioned hereinbefore.

### DEFINITIONS

The term “excipient free antibody solution” or “an antibody solution that only contains the antibody and the solvent” means an aqueous antibody-containing solution wherein small molecule solutes are only present up to a concentration of the limit of detection, for example up to a range of 0.02-0.08 mM. That is, said aqueous antibody-containing solution is essentially free of any small molecule solute above the specific limits of detection using standard analytical techniques for their detection.

The term “retentate” means the solution containing the retained protein.

The term “feed” means a solution entering the ultrafiltration cassette. During passing the semi-permeable membrane the feed is separated into the retentate and the filtrate.

The term “filtrate” means the solution passing through the membrane, containing solvent and solutes not retained by the membrane.

The term “diafiltration” means the filtration of a product with membrane filtration means with the addition of a wash fluid to the product, which causes the concentration of filterable constituents in the product to decrease, i.e., these substances are washed out without the non-filterable constituents in the product necessarily being concentrated or the product becoming thickened. Wash fluids that are used are wash fluids external to the product, such as separately supplied water or solvent.

The term “membrane ultrafiltration” means a pressure-modified, convective process that uses semi-permeable membranes to separate species in aqueous solutions by molecular size, shape and/or charge.

The term “antibody(ies)” is used herein synonymously with the term “antibody molecule(s)” and comprises, in the context of the present invention, antibody molecule(s) like full immunoglobulin molecules, e.g. IgMs, IgDs, IgEs, IgAs or IgGs, like IgG1, IgG2, IgG2b, IgG3 or IgG4 as well as to parts of such immunoglobulin molecules, like Fab-fragments, Fab'-fragments, F(ab)2-fragments, chimeric F(ab)2 or chimeric Fab′ fragments, chimeric Fab-fragments or isolated VH- or CDR-regions (said isolated VH- or CDR-regions being, e.g. to be integrated or engineered in corresponding “framework(s)”) Accordingly, the term “antibody” also comprises known isoforms and modifications of immunoglobulins, like single-chain antibodies or single chain Fv fragments (scAB/scFv) or bispecific antibody constructs, said isoforms and modifications being characterized as comprising at least one glycosylated VH region as defined herein. A specific example of such an isoform or modification may be a sc (single chain) antibody in the format VH-VL or VL-VH, wherein said VH comprises the herein described glycosylation. Also bispecific scFvs are envisaged, e.g. in the format VH-VL-VH-VL, VL-VH-VH-VL, VH-VL-VL-VH. Also comprised in the term “antibody” are diabodies and molecules that comprise an antibody Fc domain as a vehicle attached to at least one antigen binding moiety/peptide, e.g. peptibodies as described in WO 00/24782.

The antibody(ies) that may be comprised in the inventive formulation(s) are, inter alia, recombinantly produced antibody(ies). These may be produced in a mammalian cell-culture system, e.g. in CHO cells. The antibody molecules may be further purified by a sequence of chromatographic and filtration steps e.g. in order to purify specifically glycosylated antibody isoforms as described herein below.

The term “lyophilizate” as used herein in connection with the formulation according to the invention denotes a formulation which is manufactured by freeze-drying methods known in the art per se. The solvent (e.g. water) is removed by freezing following sublimation under vacuum and desorption of residual water at elevated temperature. In the pharmaceutical field, the lyophilizate has usually a residual moisture of about 0.1 to 5% (w/w) and is present as a powder or a physical stable cake. The lyophilizate is characterized by dissolution after addition of a reconstitution medium.

The term “reconstituted formulation” as used herein in connection with the formulation according to the invention denotes a formulation which is lyophilized and re-dissolved by addition of reconstitution medium. The reconstitution medium comprises but is not limited to water for injection (WFI), bacteriostatic water for injection (BWFI), sodium chloride solutions (e.g. 0.9% (w/v) NaCl), glucose solutions (e.g. 5% glucose), surfactant containing solutions (e.g. 0.01% polysorbate 20), a pH-buffered solution (e.g. phosphate-buffered solutions) and combinations thereof.

The term “stable liquid formulation” as used herein in connection with the formulation according to the invention denotes a formulation, which preserves its physical and chemical integrity during manufacturing, storage and application. Various analytical techniques for evaluating protein stability are available and reviewed in Reubsaet, J. L., J. H. Beijnen, et al. (1998). “Analytical techniques used to study the degradation of proteins and peptides: chemical instability”. J Pharm Biomed Anal 17(6-7): 955-78 and Wang, W. (1999). “Instability, stabilization, and formulation of liquid protein pharmaceuticals.” Int J Pharm 185(2): 129-88. Stability can be evaluated by storage at selected climate conditions for a selected time period, by applying mechanical stress such as shaking at a selected shaking frequency for a selected time period or by repetitive freezing and thawing at selected rates and temperatures.

The term “pharmaceutically acceptable” as used herein in connection with the formulation according to the invention denotes a formulation which is in compliance with the current international regulatory requirements for pharmaceuticals. A pharmaceutical acceptable formulation contains excipients which are generally recognized for the anticipated route of application and concentration range as safe. In addition, it should provide sufficient stability during manufacturing, storage and application. Furthermore, a formulation for a parenteral route of application should consider the requirements isotonicity and euhydric pH in comparison to the composition of human blood.

The preparation of an excipient-free antibody solution avoid excipient-induced instabilities during manufacturing and storage of an antibody solution and avoids the use of counter-ions intentionally present in the process solution or formulation. Excipients, which are usually used as additives in antibody formulations, may also contain low level of impurities which may lead to chemical instability reactions of the antibody molecule. For example, sucrose, a common used stabilizer in protein formulations, is reported to contain low traces of metal ions, which may lead to oxidation of methionine residues (Rowe R C, Sheskey P J, Owen S C. (2005) Handbook of Pharmaceutical Excipients. 5th edition ed.: APhA Publications). Furthermore, excipients may interact with surfaces of process equipment, which leads to accumulation of leachates. For example, presence of sodium chloride, also an common used isotonizer in protein formulations, was reported to increase oxidation of a therapeutic antibody at higher temperatures after contact with stainless steel surfaces (Lam et al. (1997) J. Pharm. Sci. 86(11):1250-1255).

Manufacturing of high concentrated excipient-free antibody formulations avoids the preparation of incorrect excipient compositions. Buffer-exchange at low ionic strength and high protein concentration leads to an unequal distribution of buffer-ions across the ultrafiltration membrane. Consequences of this, so called Donnan effect, are a modification of buffer concentration as a function of protein concentration and a shift of formulation pH (Stoner et al. (2004) J. Pharm. Sci. 93(9): 2332-2342). The preparation of an excipient-free antibody solution can be used as a preliminary step for preparation of a more exact antibody formulation, by adding a defined amount of a buffer stock solution to the excipient-free antibody solution.

Furthermore, this approach ensures the use of identical excipient qualities in the antibody formulation during the complete manufacturing process chain from drug substance to final drug product.

Suitable conditions for the membrane filtration can be determined by the skilled person. For diafiltration against water for injection prior concentration, the ratio of protein solution to diafiltration solution should be at least 2, more preferably at least 3 or especially preferably 5 or 10. For diafiltration and ultrafiltratio according to the invention, suitable filtrate flow rates may be in the range 1-100 L/m2h, preferably 1-80 L/m2h, in respect of the retentate and 2-60 L/m2h, preferably 3-50 L/m2h, especially preferably 8-35 L/m2h. The membrane is preferably an ultrafiltration membrane; suitable molecular weight cut-offs may be in the range 1-100 kD, preferably 5-100 kD, especially preferably 30-50 kD. The filtration may be conducted under a transmembrane pressure (TMP) in the range of 1-100 psi, preferably 10-90 psi, especially preferable 15-70 psi.

### EXAMPLES

### Example 1

A feed solution containing, a macromolecule (e.g. an antibody), solutes, such as buffer components, salts, amino acids or sugars and solvent (e.g. water) is forced by external forces (e.g. by pumping) through an ultrafiltration cassette. The feed stream is separated into a filtrate and retentate stream. The filtrate consists of the solvent and all solutes, which are able to pass the semi-permeable membrane, and leaves the system circulation. The macromolecule is retained in the retentate stream and is returned to the feed tank. During a concentration process the solvent is constantly removed and the macromolecule concentration is increased, whereas the concentration of solutes, which are able to pass the membrane, remains constant. During a diafiltration process, the discharging filtrate volume is compensated by adding diafiltration buffer to the feed tank. The diafiltration buffer consists of a different composition of solutes than the original feed solution. The concentration of the macromolecule remains constant, whereas the solute composition changes constantly from the initial feed composition to the composition of the diafiltration buffer. Both processes, concentration and diafiltration, can be combined in variable sequences.

The starting solution consisted of an IgG against the amyloid-beta peptide (Antibody A as described in Example 1 of PCT/EP2006/011914) at a concentration of approximately 50 to 60 mg/mL in 20 mM Histidine buffer. The antibody material was first pre-concentrated, then diafiltrated and subsequently concentration in the excipient-free solution to the final target concentration. The diafiltration was performed against water for injection (WFI) without further excipients. The ratio of diafiltration buffer to protein solution was at least 5. The semi-permeable membrane consists of regenerated cellulose with 400 cm2 membrane area and 30 kD MWCO. Table 5 lists an overview of process parameters obtained during the process according to the invention. Table 1 to 4 list parameters of the material such as volume (L), protein concentration (g/L), protein mass (g), pH, osmolality per g protein (mOsm/g) in the retentate, osmolality (mOsm/kg) in the filtrate, yield (%), buffer concentration (mM) as well as content of monomer (%) as determined by size-exclusion chromatography to indicate the integrity of the material after and during the process.

Osmolality per g protein in the retentate is essentially reduced throughout the process due to removal of permeable solutes. The buffer concentration was determined using a Size Exclusion (SE)-HPLC method and showed, that buffer excipients were essentially removed, below the limit of the analytical method. The absence of excipients can also indirectly be shown by osmolality values smaller than 5 mOsm/kg in the filtrate after the process.

TABLE 1
Ultra-/Diafiltration (UFDF) Run 070813A. Formulation parameter before and after ultrafiltration

| | | | | | Osmol. per | | | | |
|---|---|---|---|---|---|---|---|---|---|
| | | Protein | | | g protein | Osmol. | | Buffer | |
| | Volume | conc. | Protein | | (mOsm/g) | (mOsm/kg) | Cont. of | conc. | Yield |
| | (L) | (g/L) | mass (g) | pH | retentate | filtrate | mono. (%) | (mM) | (%) |
| Before | 0.60 | 58.9 | 35.1 | 5.6 | 0.4 | 16 | 98.1 | n.d. | n.a. |
| UFDF | | | | | | | | | |
| After | 0.31 | 111.7 | 34.9 | 5.9 | 0.1 | 0 | 97.8 | n.d. | 99.4 |
| UFDF | | | | | | | | | |

TABLE 2
UFDF Run 070814B. Formulation parameter before and after ultrafiltration

| | | | | | Osmol. per | | | | |
|---|---|---|---|---|---|---|---|---|---|
| | | Protein | | | g protein | Osmol. | | Buffer | |
| | Volume | conc. | Protein | | (mOsm/g) | (mOsm/kg) | Cont. of | conc. | Yield |
| | (L) | (g/L) | mass (g) | pH | retentate | filtrate | mono. (%) | (mM) | (%) |
| Before | 0.60 | 58.9 | 35.1 | 5.6 | 0.4 | 14 | 98.1 | n.d. | n.a. |
| UFDF | | | | | | | | | |
| After | 0.31 | 112.3 | 34.7 | 5.9 | 0.1 | 0 | 97.7 | n.d. | 98.9 |
| UFDF | | | | | | | | | |

TABLE 3
UFDF Run 070814C. Formulation parameter before and after ultrafiltration

| | | | | | Osmol. per | | | | |
|---|---|---|---|---|---|---|---|---|---|
| | | Protein | | | g protein | Osmol. | | Buffer | |
| | Volume | conc. | Protein | | (mOsm/g) | (mOsm/kg) | Cont. of | conc. | Yield |
| | (L) | (g/L) | mass (g) | pH | retentate | filtrate | mono. (%) | (mM) | (%) |
| Before | 0.60 | 58.9 | 35.2 | 5.6 | 0.4 | 16 | 98.1 | n.d. | n.a. |
| UFDF | | | | | | | | | |
| After | 0.31 | 113.6 | 34.8 | 5.9 | 0.1 | 0 | 97.8 | n.d. | 98.9 |
| UFDF | | | | | | | | | |

TABLE 4
UFDF Run 071102. Formulation parameter before and after ultrafiltration

| | | | | | Osmol. per | | | | |
|---|---|---|---|---|---|---|---|---|---|
| | | Protein | | | g protein | Osmol. | | Buffer | |
| | Volume | conc. | Protein | | (mOsm/g) | (mOsm/kg) | Cont. of | conc. | Yield |
| | (L) | (g/L) | mass (g) | pH | retentate | filtrate | mono. (%) | (mM) | (%) |
| Before | 0.56 | 50.2 | 28.0 | 5.6 | 0.4 | 13 | 95.2 | 5.0 | n.a. |
| UFDF | | | | | | | | | |
| After | 0.14 | 197.7 | 27.3 | 6.0 | 0.2 | 0 | 93.6 | 0.0 | 97.7 |
| UFDF | | | | | | | | | |

TABLE 5
Ultrafiltration process parameter

| | TMP (psi) | | | | Filtrate flow (L/m²h) | | | |
|---|---|---|---|---|---|---|---|---|
| | Conc 1 | Diafiltration | | Conc 2 | Conc 1 | Diafiltration | | Conc 2 |
| Batch | Start | Start | End | End | Start | Start | End | End |
| UFDF Run | 19.0 | 19.0 | 19.0 | 23.0 | 19.8 | 18.7 | 20.9 | 11.4 |
| 070813A | | | | | | | | |
| UFDF Run | 19.0 | 19.0 | 19.0 | 23.0 | n.d. | 18.7 | 21.2 | 10.9 |
| 070814B | | | | | | | | |
| UFDF Run | 19.0 | 20.0 | 19.0 | 24.0 | 25.4 | 21.2 | 22.1 | 12.9 |
| 070814C | | | | | | | | |
| UFDF Run | 15.0 | 16.0 | 17.0 | 25.0 | 29.5 | 25.7 | 28.0 | 4.3 |
| 071102 | | | | | | | | |

### Example 2

The starting solution consisted of an IgG antibody against VEGF at a concentration of 44.6 mg/mL in phosphate buffer. The IgG antibody against VEGF is described in US 2008/0248036 A1. This anti-VEGF antibody “Bevacizumab”, also known as “rhuMAb VEGF” or “Avastin™”, is a recombinant humanized anti-VEGF monoclonal antibody generated according to Presta et al. (1997) Cancer Res. 57:4593-4599. It comprises mutated human IgG1 framework regions and antigen-binding complementarity-determining regions from the murine anti-hVEGF monoclonal antibody A.4.6.1 that blocks binding of human VEGF to its receptors. Approximately 93% of the amino acid sequence of Bevacizumab, including most of the framework regions, is derived from human IgG1, and about 7% of the sequence is derived from the murine antibody A4.6.1. Bevacizumab has a molecular mass of about 149,000 daltons and is glycosylated. Bevacizumab is being investigated clinically for treating various cancers, and some early stage trials have shown promising results. Kerbel (2001) J. Clin. Oncol. 19:45 S-51S; De Vore et al. (2000) Proc. Am. Soc. Clin. Oncol. 19:485a; Johnson et al. (2001) Proc. Am. Soc. Clin. Oncol. 20:315a; Kabbinavar et al. (2003) J. Clin. Oncol. 21:60-65.

The antibody material was first pre-concentrated, then diafiltrated and subsequently concentration in the excipient-free solution to the final target concentration. The diafiltration was performed against water for injection (WFI) without further excipients. The ratio of diafiltration buffer to protein solution was at least 5. The semi-permeable membrane consists of regenerated cellulose with 400 cm2 membrane area and 30 kD MWCO. Table 7 lists an overview of process parameters obtained during the process according to the invention. Table 6 list parameters of the material such as volume (L), protein concentration (g/L), protein mass (g), pH, osmolality per g protein (mOsm/g) in the retentate, osmolality (mOsm/kg) in the filtrate, yield (%), buffer concentration (mM) as well as content of monomer (%) as determined by size-exclusion chromatography to indicate the integrity of the material after and during the process.

Osmolality per g protein in the retentate is essentially reduced throughout the process due to removal of permeable solutes. The absence of excipients can also indirectly be shown by osmolality values smaller than 5 mOsm/kg in the filtrate after the process.

TABLE 6
UFDF Run 071106. Formulation parameter before and after ultrafiltration

| | | | | | Osmol. per | | | | |
|---|---|---|---|---|---|---|---|---|---|
| | | Protein | | | g protein | | Osmol. | Buffer | |
| | Volume | conc. | Protein | | (mOsm/g) | Cont. of | (mOsm/kg) | conc. | Yield |
| | (L) | (g/L) | mass (g) | pH | retentate | mono. (%) | filtrate | (mM) | (%) |
| Before | 0.56 | 44.6 | 25.2 | 6.2 | 2.8 | 94.4 | 86 | n.d. | n.a. |
| UFDF | | | | | | | | | |
| After | 0.11 | 209.6 | 22.0 | 6.6 | 0.1 | 84.8 | 3 | n.d. | 87.6 |
| UFDF | | | | | | | | | |

TABLE 7
Ultrafiltration process parameter

| | TMP (psi) | | | | Filtrate flow (L/m²h) | | | |
|---|---|---|---|---|---|---|---|---|
| | Conc 1 | Diafiltration | | Conc 2 | Conc 1 | Diafiltration | | Conc 2 |
| Batch | Start | Start | End | End | Start | Start | End | End |
| UFDF Run | 18.0 | 18.5 | 18.5 | 69.0 | 9.9 | 8.8 | 17.7 | 3.1 |
| 071106 | | | | | | | | |

### Example 3

The starting solution consisted of an IgG antibody against MUC1 (cell surface associated mucin 1) at a concentration of 10.2 mg/mL in 20 mM acetate buffer containing sodium chloride. This antibody is described for example in (i) Taylor-Papadimitriou J, Peterson J A, Arklie J, Burchell J, Ceriani R L, Bodmer W F 1981. Monoclonal antibodies to epithelium-specific components of the human milk fat globule membrane: production and reaction with cells in culture. Int J Cancer 28(1):17-21 and (ii) in Verhoeyen M E, Saunders J A, Price M R, Marugg J D, Briggs S, Broderick E L, Eida S J, Mooren A T, Badley R A 1993. Construction of a reshaped HMFG1 antibody and comparison of its fine specificity with that of the parent mouse antibody. Immunology 78(3):364-370.

The antibody material was first pre-concentrated, then diafiltrated and subsequently concentration in the excipient-free solution to the final target concentration. The diafiltration was performed against water for injection (WFI) without further excipients. The ratio of diafiltration buffer to protein solution was at least 5. The semi-permeable membrane consists of regenerated cellulose with 400 cm2 membrane area and 30 kD MWCO. Table 9 lists an overview of process parameters obtained during the process according to the invention. Table 8 list parameters of the material such as volume (L), protein concentration (g/L), protein mass (g), pH, osmolality per g protein (mOsm/g) in the retentate, osmolality (mOsm/kg) in the filtrate, yield (%), buffer concentration (mM) as well as content of monomer (%) as determined by size-exclusion chromatography to indicate the integrity of the material after and during the process.

Osmolality per g protein in the retentate is essentially reduced throughout the process due to removal of permeable solutes. The buffer concentration was determined using an Reversed Phase (RP)-HPLC method and showed, that buffer excipients were essentially removed. The absence of excipients can also indirectly be shown by osmolality values smaller than 5 mOsm/kg in the filtrate after the process.

TABLE 8
Batch UFDF Run 071108. Formulation parameter before and after ultrafiltration

| | | | | | Osmol. per | | | | |
|---|---|---|---|---|---|---|---|---|---|
| | | Protein | | | g protein | | Osmol. | Buffer | |
| | Volume | conc. | Protein | | (mOsm/kg) | Cont. of | (mOsm/kg) | conc. | Yield |
| | (L) | (g/L) | mass (g) | pH | retentate | mono. (%) | filtrate | (mM) | (%) |
| Before | 2.53 | 10.2 | 25.8 | 6.0 | 26.1 | 99.3 | 244 | 27.5 | n.a. |
| UFDF | | | | | | | | | |
| After | 0.11 | 203.3 | 23.2 | 6.1 | 0.2 | 99.2 | 1.0 | 4.4 | 89.9 |
| UFDF | | | | | | | | | |

TABLE 9
Ultrafiltration process parameter

| | TMP (psi) | | | | Filtrate flow (L/m²h) | | | |
|---|---|---|---|---|---|---|---|---|
| | Conc 1 | Diafiltration | | Conc 2 | Conc 1 | Diafiltration | | Conc 2 |
| Batch | Start | Start | End | End | Start | Start | End | End |
| UFDF Run | 19.5 | 23.0 | 23.0 | 41.0 | 33.3 | 12.8 | 31.7 | 6.3 |
| 071108 | | | | | | | | |

While there are shown and described presently preferred embodiments of the invention, it is to be distinctly understood that the invention is not limited thereto but may be otherwise variously embodied and practiced within the scope of the following claims.

1. A method of ultra- and diafiltrating an antibody solution containing at least one solute in addition to the antibody, which comprises diafiltering the antibody solution with a solvent and bringing said mixture into contact with a semi-permeable membrane so as to allow the at least one solute present in the antibody solution and having a molecular weight lower than the molecular weight cut-off of the membrane to pass through the membrane, whilst retaining the antibody so that an antibody solution is obtained that only contains the antibody and the solvent.

2. A method as claimed in claim 1, wherein the solvent is water.

3. A method as claimed in claim 1, wherein said antibody solution that only contains the antibody and the solvent has an antibody concentration of from 30 to 280 mg/mL, preferably of from 50 to 200 mg/mL.

4. A method as claimed in claim 1, which further comprises the step of processing said antibody solution that only contains the antibody and the solvent to a stable liquid formulation.

5. A method as claimed in claim 1, which further comprises the step of processing said antibody solution that only contains the antibody and the solvent to a lyophilizate or reconstituted formulation.

6. A method as claimed in claim 1, wherein the at least one solute is selected from the group consisting of buffer salts, salts, amino acids, sugars and sugar alcohols.

7. A method as claimed in claim 1, wherein the semipermeable membrane is an ultra-filtration membrane, preferably having a molecular weight cut-off in the range of 2-50 kD.

8. A method as claimed in claim 1, wherein the antibody is a monoclonal antibody, preferably selected from the group of IgG1, IgG2 or IgG4.

9. A purified antibody solution obtainable by a method as claimed in claim 1.

10. A purified antibody solution as claimed in claim 1, wherein the antibody is a monoclonal antibody, preferably selected from the group of IgG1, IgG2 or IgG4.

11. The method and purified antibodies substantially as hereinbefore described, especially with reference to the foregoing examples.
